(19) United States
(12) Patent Application Publication
(10) Pub. No.: US 2010/0035295 A1
(43) Pub. Date: Feb. 11, 2010

(54) Systems to analyze cellular metabolism and cell and molecular reactions

(76) Inventor: Patricia J. Malin, Palo Alto, CA (US)

Correspondence Address: Patricia J. Malin, 755 Page Mill Road, Palo Alto, CA, 94304, US

(21) Appl. No.: 12/462,534

(22) Filed: Aug. 5, 2009

Related U.S. Application Data
(60) Provisional application No. 61/188,321, filed on Aug. 9, 2008.

(51) Int. Cl. C12Q 1/02; C12M 1/34

(52) U.S. Cl. 435/29; 435/287.1

(57) ABSTRACT

The present invention relates to systems capable of measuring an electrical signal produced by a cell, for example, by the metabolic activity of a cell. A system of the invention relates to varying the applied voltage and frequency to a culture vessel containing cells to account for the differences in cell metabolism of various and numerous cell types. A system of the invention, in certain embodiments, comprises a measurement board and a microprocessor. In certain other embodiments, the invention relates to methods for analyzing a cell or tissue using a system of the invention.

### 1.0 FIELD OF THE INVENTION

The present invention relates to systems to analyze the metabolism of cells along with cell cycle phases, gene functions, and biochemical reactions and molecular reactions. The present invention also relates to the device that reads the various signals that are produced by the cells' metabolic activity, cell cycle phases, gene functions, biochemical reactions, and molecular reactions. These signals differ based on the cell type, the cells metabolic rate, the size of the cell and the cells proliferation rate. The present invention also relates to being able to take measurements rapidly, in microseconds or nanoseconds and to constantly calibrate the hardware in order to read the cells' signals with precision

### 2.0 BACKGROUND

Cells of living organisms engage in biochemical metabolism in which large numbers of reactions of many kinds take place. While the numbers are not precisely known, it has been estimated that typically, over a period of 24 hours, many chemical reactions take place in a single cell, for example, as many as 100 to 1 billion. These reactions happen in nanosecond or even in picoseimen time. Cellular metabolism differs depending on various parameters, for example, the type of cell and whether the cell is healthy or diseased, and which disease. The ability to measure cellular metabolism with high speed and accuracy, and preferably under controlled conditions so that one may compare results of different measurements, would be useful for multiple applications in research, diagnosis, and therapy. The present invention provides systems that acquire the signals produced by a cellular event, and facilitates analysis of those signals to monitor cellular metabolism. In addition, the present invention is capable of monitoring reactions within the cell or cells as they occur and in nanosecond time. Being able to examine overall cellular metabolism as well as biochemical or molecular reactions, as they occur, within a cell will be beneficial in both research and clinical medicine.

### 3.0 SUMMARY OF THE INVENTION

A system of the current invention, in certain embodiments, is capable of measuring an electrical signal produced by a cell, for example, by the metabolic activity of a cell including specific metabolic processes or reactions. A system of the invention, in certain embodiments, comprises a measurement board and a microprocessor, preferably a soft microprocessor. In a preferred embodiment, a system of the invention comprises a soft microprocessor built into a measurement board. In certain embodiments, a system of the invention comprises an Ethernet communication, a LCD display, an EEPROM configuration, a remote GUI interaction, a relay matrix management, a temperature control switch, a CO₂ sensing subsystem, and/or an O₂ sensing subsystem. In certain other embodiments, the invention relates to methods for analyzing a cell or tissue using a system of the invention.

In certain embodiments of the system, the software program can set both the applied frequency and applied voltage. In other embodiments of the system, an array of applied frequency and applied voltages can be applied to the probes. This is important since the metabolic activity of cells and therefore the signals produced are dependent upon the cell type. For example, the metabolic activity of cancer cells is different than the metabolic rate of normal cells. In addition, the metabolic activity of cancer cells varies depending upon the aggressiveness of the cancer. In another embodiment of the system by shortening the timing interval between scans, the system can be used to determine the mitotic stages of the cell.

In certain embodiments a system is comprised of both software and hardware modules that allow for precision control of the environmental chamber, constant calibration of the devices, hardware to minimize any noise in the system, and rapid readings of cellular events. In the current embodiment, two probes are placed inside the culture dish or well and a charge is placed on one of the probes, the other being a ground. In other embodiments, planar electrodes or placing the probes directly inside a cell can be used. The invention in certain embodiments utilizes an end user computer software program that gives instructions to the system on the board. The board has two components a computer and a relay matrix network

### 4.0 BRIEF DESCRIPTION OF THE DRAWINGS

FIG. 1: A block diagram of a system according to certain embodiments of the invention is shown.

FIG. 2: A diagram illustrating the operation of a system according to certain embodiments of the invention.

FIG. 3: A diagram illustrating software of a system according to certain embodiments of the invention.

FIG. 4: An embedded firmware block diagram of a system according to certain embodiments of the invention.

FIG. 5: A graphical human interface block diagram of a system according to certain embodiments of the invention.

FIG. 6: A block diagram of the three main components of a system according to certain embodiments of the invention.

### 5.0 DETAILED DESCRIPTION OF THE INVENTION

The present invention provides systems capable of measuring an electric signal of a cell. A system of the current invention, in certain embodiments, is capable of taking a measurement in a short period of time, for example, in the nanosecond or picosecond range. In certain embodiments, a system of the invention applies a voltage to a cell being analyzed and in certain embodiments, the voltage may be changed in magnitude and/or frequency, for example, to detect different signals from the cell. In certain other embodiments, a system of the invention is capable of monitoring and/or maintaining the level of CO₂ and/or O₂ of an environment of a cell (for example, in a cell incubator) that is being analyzed with the system. In another embodiment of the system, any variable within the environmental chamber that is capable of being monitored can be controlled.

In certain embodiments, a system of the invention comprises a soft microprocessor built into a measurement board (or board) of the system. In certain embodiments, a system of the invention comprises a hardware controlled by software that monitors, preferably continuously, the hardware and/or software, for example, to maintain its functionality.

A system of the current invention, according to certain embodiments, applies a small charge, using two probes that are inserted into culture vessels with a liquid suspension that holds the cell specimen and then reads a return charge (the signal). In certain embodiments the culture vessel can hold a single cell or many cells such as would be found in a bioreactor or fermentor. The signal is in part the result of a measurable electrical charge of the cell or cells. The changes in this signal over time have a demonstrable and repeatable correlation to changes in cell metabolism. Such signals are a characteristic of a state of a cell, for example, the extent of its activation, its metabolism, its differentiation, etc. As these signals are acquired in real-time, a system of the invention in certain embodiments allows monitoring changes of a state of the cells during proliferation or differentiation, or in response to growth factors or cytokines, or as modulated by drugs and other treatments and conditions (for example, disease progression). Measuring signals of a cell over time allows, for example, the observation of effects that may be temporal and that could be missed if only detecting an end-point past these changes.

In other embodiments monitoring the electric voltage changes of 1-10 cells that have been synchronized to go through the cell cycle simultaneously would be beneficial. This could either be done in a liquid suspension or by applying the probes directly into the cells.

A system of the current invention, according to certain embodiments, uses a line conditioner and/or a voltage regulator for electricity coming into the system, for example, to reduce or preferably eliminate potential electronic interferences from outside sources with a signal detected from a cell. In certain other embodiments, a system of the invention comprises a software program to monitor and/or calibrate hardware of the system to maintain functionality, for example, to reduce or eliminate drift and/or noise that may occur at times in a measurement hardware of a system of the invention.

A system of the current invention, according to certain embodiments, allows adjustment of a charge applied to a cell (for example, its voltage) and the frequency with which a charge is applied to enable the monitoring of specific cell signals. A system of the current invention, according to certain preferred embodiments, allows data acquisition with high speed, for example, detecting data that occur in nanoseconds and/or processing a signal produced in such a nanoseconds time-range. For example, each data point may be taken in the range of 100 to 200 nanoseconds depending on the experimental set-up. For example, this could mean that 96 wells could be monitored from anywhere between 9.6 to 19.2 microseconds. This could make it possible, for example, to take readings of biochemical events occurring within the cell as they occur.

A system of the current invention, according to certain embodiments, allows taking measurements with high speed and therefore the time between two measurements can be short. For example, two measurements may be 100 nanoseconds apart, or 500 nanoseconds, or 1000 nanoseconds, of 2000 nanoseconds, or 4000 nanoseconds, or 100 to 4000 nanoseconds. In certain embodiments, a system of the current invention takes measurements in close succession to facilitate detection of changes in a cell or an organelle in a cell, for example, fluctuations and/or interactions within a cell caused by an organelle, a gene, a protein, a signaling event, or anything else. Understanding and modeling empirically the fluctuations and interactions of biochemical reactions within a cell assists in analyzing and understanding gene circuits, networks, and different cellular pathways. A device of the current invention, in certain embodiments, may also identify a signal occurring outside a set range so that one may determine if a signal is significant or an artifact.

A system of the current invention, according to certain embodiments, controls the environment of a cell that is analyzed with the system. For example, a system of the invention may control settings of a cell incubator in which a cell that is analyzed with the system is kept, for example, temperature, CO₂, O₂, humidity, and/or any other parameter. A system of the invention, in certain embodiments, may facilitate directly or indirectly controlling one or more of temperature, pH, and/or redox environment of a cell that is analyzed with the system. Cells, including mammalian cells (for example, cells from human or mouse), typically require a constant or substantially constant temperature (for example, 37.5° C. (Celsius)) and/or extracellular pH (for example, about 7.2) for optimal growth. Also, in culture, a bicarbonate-carbon dioxide buffering system may be used to maintain pH. Although many culture systems utilize atmospheric oxygen (final concentration approximately 20% oxygen), physiological oxygen concentrations needed in a cellular environment are often lower (for example, ranging from 2-8%), so that an ability to control oxygen in a cell culture system is desirable. A change in a variable in a cellular environment may impact biochemical reactions and/or overall cellular metabolic activity. Such a change may also impact a voltage produced by metabolic activity of a cell. Therefore, it is desirable, when reading a small change in the voltage that a cell produces using a system of the invention, to control the cellular environment, preferably with a degree of precision that helps to reduce and preferably eliminate the impact of those changes on the data acquired.

A system of the current invention, according to certain embodiments, comprises one or more sensors for CO₂, O₂, humidity, and/or temperature of the environment of a cell that is analyzed with the system. One or more microprocessors and software of a system of the invention, in certain preferred embodiments, monitor the cellular environment, adjust the levels of CO2 and O2, coming into the cellular environment, and maintain the temperature of the environment at a constant level. Preferably, a system of the invention carries out the monitoring, adjusting, and maintaining of the environment of a cell in a continuous manner or with sufficient frequency to facilitate an analysis as desired. Preferably, someone using a system of the invention sets a desired level of one or more environmental parameter; and a computer and/or hardware maintains it. If identical experiments are being performed at different locations, a system of the invention in certain embodiments facilitates calibrating electronics used in the device and to standardize the cellular environment at the different locations. A system of the invention in certain embodiments may utilize a LAN or it may operate over the Internet. A system of the invention, in certain embodiments, can be used as an individual unit with the same capabilities to maintain the cellular environment or the hardware as discussed herein.

### 5.1 Configurations of Systems of the Invention

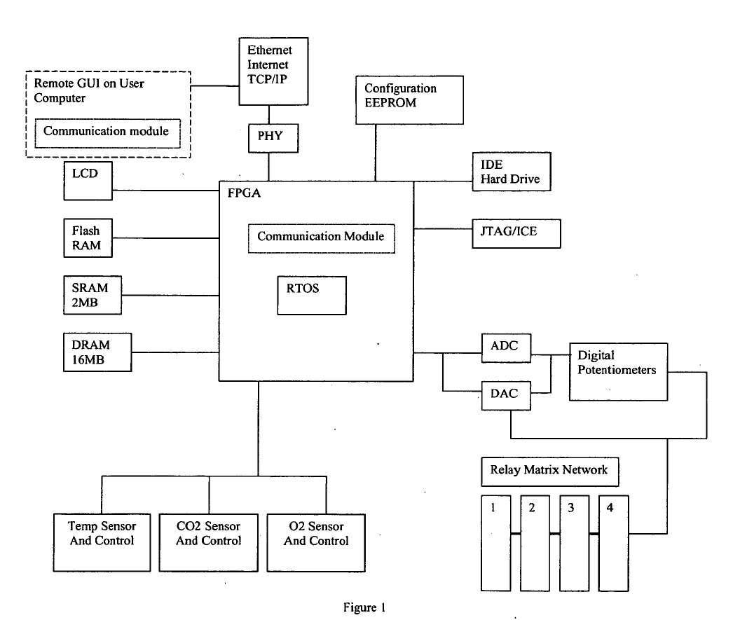

Systems according to certain embodiments of the invention and various aspects of those systems are illustrated in the figures. FIG. 1 shows a block diagram of a system according to certain embodiments of the invention. Components discussed and abbreviations used herein include the following: A flash memory refers to a non-volatile memory that can be electronically erased and reprogrammed such as Intel's Advanced & Boot Block Flash memory. An EEprom is an electronically erasable programmable read only memory. JTAG/ICE refers to on-chip debugging. A PHY chip (also called PHYceiver) can be found on Ethernet devices and its purpose may be digital access of a modulated link. A FPGA refers to a field-programmable gate array that is a semiconductor device containing programmable logic. A SDRAM means synchronous dynamic random access memory which is a type of solid-state computer memory. A (SRAM) static random access memory is a type of semiconductor memory where the word static indicates that it does not need to be periodically refreshed, unlike dynamic RAM (DRAM), as SRAM uses bi-stable latching circuitry to store each bit. SRAM exhibits data remanence but is still volatile in the conventional sense that data is eventually lost when the memory is not powered.

In a system shown in FIG. 1 are two main sections. One section is the computer with the FPGA and soft microprocessor running a real time operating system (RTOS) and the other section is the Relay Matrix Network. In a system shown in FIG. 1, a main component is a FPGA (for example, the Xilinx Spartan FPGA) that controls the function of the measurement board. Inside the FPGA, one may use a 32-bit processor (for example, a RBT-32 (Royal Bengal Tiger)) running a real-time operating system (for example, TUWA™ that may be programmed for fast data acquisition). Instructions for the processor are preferably programmed for real-time data acquisition. The FPGA illustrated in FIG. 1 preferably manages all or substantially all devices, for example, Ethernet communication, LCD display, EEPROM configuration, remote GUI interaction, relay matrix management, temperature control switch, CO₂ sensing subsystem, and/or O₂ sensing subsystem. In certain embodiments, an FPGA of a system of the invention creates interfaces with some or all of the devices that interact with RBT-32. These interfaces are: a) Oxygen sensor; b) CO2 Sensor; c) EEprom; d) A/D conversion interface; e) D/A conversion interface; f) 8 MB FLASH memory; g) 2 MB of static RAM; h) 16 MB of DRAM; i) IDE controller for hard-disk attachment; j) 4 Serial ports; k) 1 parallel port; and/or l) LCD controller (for incubator front panel).

In certain embodiments there are embedded firmware modules in the FPGA that control the various functions of the system. The embedded system control module located in the FPGA. The embedded system control module controls the embedded firmware service modules. In this embodiment the system control module is the heart of the firmware; it controls all the operation of the analyzer. It routes user commands, data, and status to all other modules of the firmware.

### 5.2 Operations of Systems of the Invention

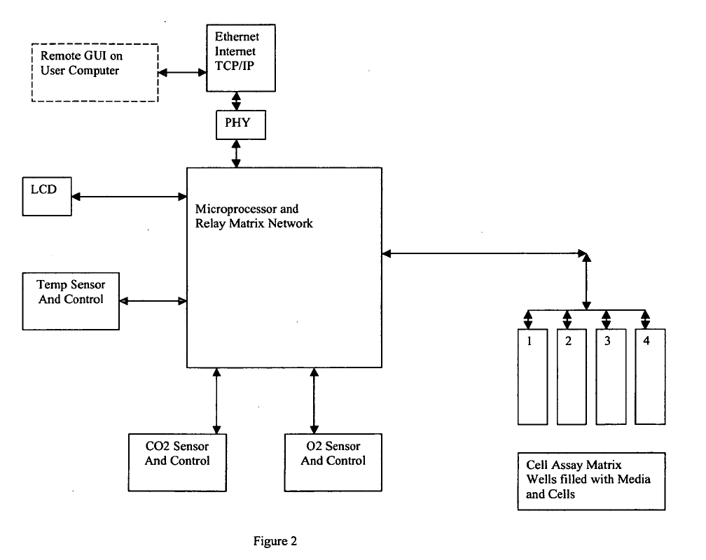

FIG. 2 illustrates operational aspects, some or all of which may be found in a system according to certain embodiments of the invention. A graphical user interface from the user computer transmits the variables of the test for the operation of the metabolic analyzer of the system. A user may create an analysis profile in the local computer. When the analysis profile of a user is run, the user program sends the configuration information to the board for that particular profile, for example, temperature, CO₂, O₂, scan intervals, and/or a magnitude for a stimulus to be applied to cells, or any other configuration information. The board may be controlled through a TCP/IP network. After receiving a configuration, the board may run a data acquisition as desired, it may store the data on the local DRAM or transmit them back to the user computer. A real-time operating system (RTOS) may interact with a remote user computer. An RTOS may sense all required sensors and it may manage them in accordance with an analysis profile. RTOS may also program the FPGA so that it can generate a proper stimulus voltage that is applied to a cell analyzed with the system. RTOS may also facilitate that the stimulus voltage is correctly applied by reading back the voltage at the relay contact points. EEprom may save the board configuration, for example, ip and/or mac address of the board, incubator control information (for example, for the last run before the door was open and any or all other last run configurations), and/or in case of failure, a possible cause of failure, or any other configuration.

A flash device may be used to store the RTOS and data acquisition application. A DRAM and a static RAM may be used for RTOS and application data processing. An LCD device may be used to show a current temperature, a current run, and/or a company logo on the incubator front. A D/A conversion device may be used to take the input from the FPGA as digital numeric number of the stimulus voltage and it may convert it to the analog output voltage. In order to apply an AC type signal, RTOS may send a varying number in the DA input so that an analog output varies like an AC signal. An A/D conversion device may read the relay voltage in the analog form and then convert it into the digital format. An FPGA may read a digital output from an A/D conversion device. These digital samples may be the actual scanned data. An RTOS may read these scanned data and process them before it sends them back to the user computer.

The measurement board of a system of the invention may have one or more electronic relay switches (or relay(s)). A relay may be controlled by the logic in the FPGA. A RTOS may drive an address and/or a data line of a 32 bit processor (e.g., a RBT-32) that may drive the relay logic for proper data acquisition from wells in which cells are located that are being analyzed with a system of the invention.

A FPGA of a system of the invention may comprise a connector (e.g., a 40 pin connector), which may be connected to a hard disk where data and profiles may be saved. An IDE controller may be designed inside the FPGA using, for example, Verilog HDL language. One or more serial ports (e.g., four serial ports) may be created in the FPGA, for example, using Verilog HDL language. The serial ports may be used for debugging the board, for configuring the board, and/or for connecting a CO₂ sensor.

One or more parallel ports may be used, for example, so that a printer can be added. A parallel controller may be designed, for example, by using Verilog HDL. Software of a system of the invention may be written in C/C++ and/or assembly language. Some or all of the hardware IPs may be written in Verilog HDL language.

### 5.3 Configurations of Software in Systems of the Invention

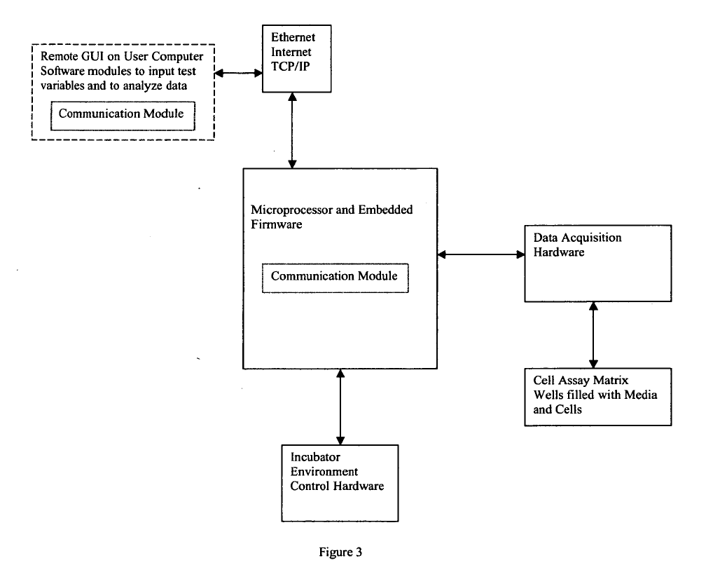

The software of a system of the invention, in certain embodiments, may comprise one, two, or more sections. FIG. 3 illustrates software and software relationships, some or all of which may be found in a system according to certain embodiments of the invention, including a software section comprising embedded firmware and a software section comprising graphical human interface for operation, control and/or data analysis.

### 5.3.1 Embedded Firmware

Embedded firmware is a basic component of a cell metabolic analyzer system according to certain embodiments of the invention. Embedded firmware may receive control and/or configuration commands from the user over the TCP/IP link. A packet protocol may be used to carry some or all control and/or configuration information regarding the system according to certain embodiments. According to certain embodiments, when embedded firmware receives a command through a network link, a communication module may pass such a command to a system control module. The system control module, according to these embodiments, may then interpret the command and route the command to a command service module.

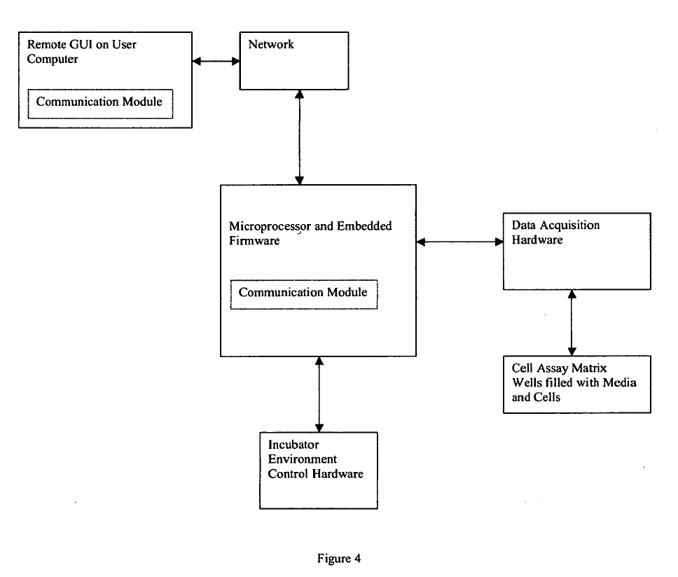

FIG. 4 illustrates modules of an embedded firmware of a system of according to certain embodiments of the invention comprising a communication module, a system control manager, a sensor monitor, an incubator control module, a data acquisition subsystem, and/or a configuration manager. A communication module according to certain embodiments exchanges data packets over the TCP/IP network. The communication module located in the embedded firmware may receive control and configuration messages from the communication module on a User GUI program, and/or it may send back status and data information to GUI. In a system according to these embodiments, this is an important path to control the metabolic analyzer.

A system control manager according to certain embodiments is a basic component of the firmware. A system control manager may control all or most of the operation of the analyzer according to certain embodiments. A system control manager may route user commands, data, and/or status information to some or all of the other modules of the firmware.

A sensor monitor according to certain embodiments monitors one or more environment sensors and sends information to the system control manager. The system control manager according to certain embodiments may take one or more decisions on whether to adjust the environment of the cells that are or may be analyzed and it may send one or more messages to a control module for the incubator of the cells that are being analyzed or may be analyzed with the system of the invention. The system control manager may also send a status message to GUI over the TCP/IP link.

A system of the current invention, according to certain embodiments controls an incubator in which the cells to be analyzed by the system of the invention are kept. Such control may comprise controlling one, two, three, four, or more parameter of the incubator, for example, temperature, CO₂, O₂, or any other parameter of interest. The incubator embedded firmware module monitors all information pertaining to the incubator, such as the last run before the incubator door was opened, and all other last run configuration information. It monitors whether or not the incubator has a failure and identifies the possible cause of that failure. It can transmit over the embedded communications module information to the systems control module. The systems control module will then transmit that information over the TCP/IP to the GUI on the host computer. It will also make any necessary adjustments to the incubator. These built in control systems and information will help the researcher or clinician identify any discrepancies in the data due to any problem for example, a power failure.

A data acquisition subsystem according to certain embodiments receives one or more data read requests from a system control manager and/or it may control data acquisition hardware. A configuration manager according to certain embodiments may save equipment operating configuration in a non-volatile memory and/or during power recycle some or all equipment may read the configuration information and the equipment is configured.

Embedded firmware of a system of the invention according to certain embodiments may run a real-time environment on TUWA RTOS customized for the data acquisition system of the invention.

### 5.3.2 Graphical Human Interface

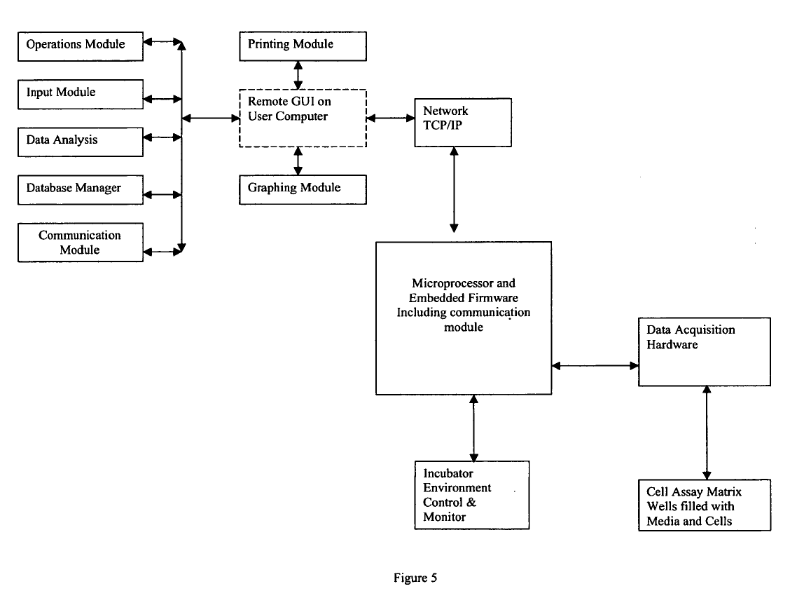

FIG. 5 illustrates a graphical human interface according to certain embodiments of the invention. A system of the current invention according to certain embodiments comprises a graphical human interface for operation, for control and/or for running data analysis software. A communication module according to certain embodiments sends and/or receives command and/or data packets to a data acquisition board, for example, a real-time data acquisition board.

Experimental variables and input measurement variables including frequency and voltage amplitude are also transmitted from GUI to the data acquisition embedded module located in the FPGA. The program has a default frequency and amplitude but different values may be set for a specific experiment or test.

An environment control and monitor according to certain embodiments monitors an incubator for cells, for example, temperature, CO₂, O₂, or any other parameter of interest. The environment control and monitor may send control information to an embedded board for adjustment of the environment of the cells that are or may be analyzed with a system of the invention. A graphing GUI according to certain embodiments draws various graphs of data obtained using a system of the invention or data considered during operation of the system.

A GUI in certain embodiments may display data acquisition steps and/or process mouse clicks on button. An experiment setup GUI of a system of the invention in certain embodiments sets up some or all aspects of the information for and/or related to an experiment. Some or all of such information may be stored in a database. In certain embodiments, a database manager may store and/or retrieve information from the database. In certain other embodiments, a printing service may print graphs and/or reports.

In another embodiment the graphical interface has additional components that maintains the database of all data acquired, can plot, graph analyze and report that data.

In another embodiment, the graphical interface can also control all devices used in an experiment over a LAN or Internet. It can control devices at any global location, calibrate, and standardize them to a single set of variables. In another embodiment the user interface can search for data in research articles on the Internet, grade them according to their importance, and bring them into the database. In another embodiment of the invention experimental data from other standard assays used i.e. calorimetric, fluorescence, molecule and biochemical assays can be brought into the database for use in analyzing and comparing different sets of data. In another embodiment, researchers and clinicians, using different systems of the device, will be able to communicate and discuss the same type of data from experiments or diagnostic information.

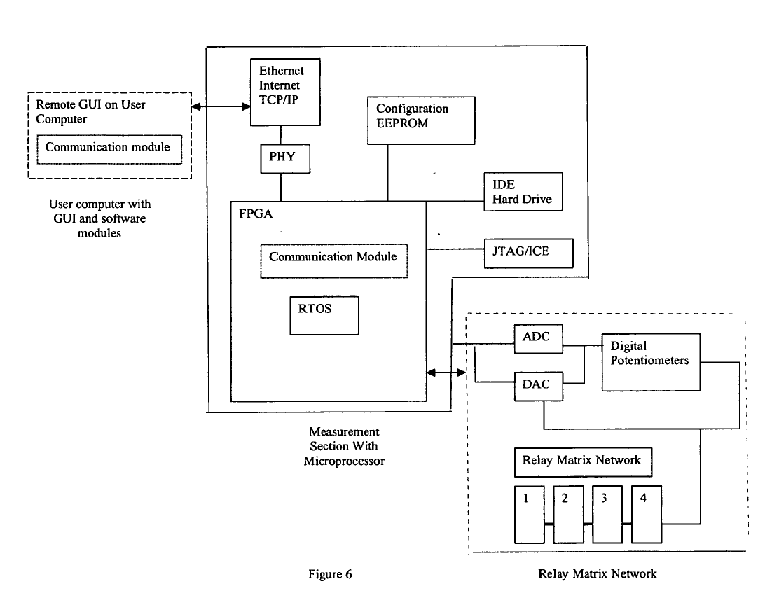

FIG. 6 illustrates the three main sections of a system according to certain embodiments. The end user GUI computer with software modules that include but are not limited to communication, data analysis and information technology. The measuring device with embedded firmware modules that include, but are not limited to, data acquisition, sensor control, and communication. The Relay Matrix Network that includes but is not limited to the hardware used for the cell-based and molecular based application.

### 5.4 Taking Measurements

In certain embodiments, a system of the current invention may take a measurement as follows. Once a user has configured an experiment setup and started an experiment by clicking the button, the software will calibrate all the data acquisition hardware, and check to make sure the data acquisition software is performing as specified. The GUI software then may generate a read data command. The command will use a well number (on the cell culture plate, for example, a 96-well plate) and send the well number and the read data command to the acquisition board over the TCP/IP link. When the board receives a command, it will send the information to the data acquisition module. This module will send a command to hardware logic, for example, written in Verilog. This hardware logic will select a well, apply a signal of desired frequency and amplitude, and read a signal from the well. It will also cancel any unwanted signal levels and/or present the data to the data acquisition module. In other embodiments, unusual signal levels will be marked so that they can be viewed to determine whether the signal is significant or unwanted. Illustrations of an unwanted signal would be the opening and closing of the environmental chamber door. Such an event, the opening or closing of the door to the environmental chamber, would be maintained in the log activity and the signal could be checked against the log to determine if the event, the opening and closing of the environmental chamber door, caused the unwanted signal. This module may also normalize and pre-process data more before sending it back to the GUI for viewing.

### 5.5 Uses of Systems of the Invention

A system of the invention according to certain embodiments may be used to analyze cells and/or tissues of various types, for example, fibroblasts, epithelial cells, muscle cells, nerve cells, neurons, glia cells, chondrocytes, stem cells, embryonic stem cells, progenitor cells, hematopoietic stem cells, blood cells, immune cells, or any other cell and/or tissue.

A system of the invention according to certain embodiments may be used to analyze cells and/or tissues involved in various diseases, for example, cancer, brain cancer, colon cancer, breast cancer, prostate cancer, skin cancer, lung cancer, liver cancer, leukemia, cervical cancer, lymphoma, melanoma, immune disorders, diabetes, chronic disorders, muscular dystrophy, Alzheimers, Parkinsons, HIV, cystic fibrosis, autoimmune diseases, allergies, lupus, infectious diseases, viral infectious diseases, bacterial infectious diseases, kidney diseases, liver diseases, thyroid diseases, hormonal disorders, blood disorders, bone diseases, gastrointestinal diseases, and/or any other disease.

Examples of applications of a system of the invention according to certain embodiments comprises discerning voltage changes caused by altered biological processes, for example, determine if a cell sample is cancerous, determine the type of cancer, determine the stage of cancer, determine the aggressiveness of a cancer type, determine the presence of analytes or genetic markers using analyte specific reagents that are electrically variable in the presence of the analyte, determine the health status of normal cells including responsiveness to physiological stress, determine changes in the phenotype of the cell sample, determine bacterial contamination of food, cell counting or density for pharmaceutical use, cell counting or density for clinical use, determine the best time to administer a therapeutic, determine bacterial contamination in patient sample, determine bacterial contamination in non-patient sample (e.g., meat), determine antimitotic response, determine apoptosis, determine bacterial growth, determine cancer proliferation rate, determining cytotoxicity of a drug, determine best drug cancer treatment, determine allergic response to growth factors, growth kinetics, hyperplasia (abnormal increase in growth of normal cells), grow hemopoetic cells for patient treatment, industrial cell culture monitoring for antibodies, measuring a non mitotic response—hypertrophy, determine best serum free media for vaccines, use as a quality control assay, and/or determine viral contamination.

Other examples of applications of the system are to study cell cycle phases and the differences in the cell cycles of cancer vs. normal cells. Also to study the impact of a Gene (protein) of different cell types that has different genetic material.

The monitoring of cellular activity over a specific period from a minute to fourteen plus days gives information that has never been accessible.

While in the current embodiment it is being used to control and measures a cell analysis device. In another embodiment the embedded system control module and other aspects of the embedded firmware can be used to constantly calibrate and monitor other types of systems such as neonatal incubators or air flaps on airplane wings.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited publications, patents, and patent applications are herein incorporated by reference in their entirety for any purpose.

What is claimed is:

1. A device for analyzing a cell or cells comprising a microprocessor, a measurement board, a user interface, and software for operating said device, wherein said microprocessor is integrated with said measurement board, wherein said software is capable of controlling a parameter or parameters in an environment of the cell, and wherein said device can be calibrated over the internet.

2. The device from claim #1 that also constantly takes measurements of picoseimen or smaller changes in as little as little as nanosecond time.

3. A method of taking measurements and controlling all firmware and hardware eliminating the need for interrupts in the embedded firmware thereby allowing for simultaneously control of all processes and increasing the speed that the data can be acquired.

4. The method from claim #3 that also constantly takes measurements of picoseimen or smaller changes in as little as little as nanosecond time.

5. The method from claim #3 also is a method of generating different frequencies by utilizing the RTOS to send varying numbers to the digital analog converter thereby increasing the range of the frequency that can be generated

6. The method from claim #3 also is a method of using the RTOS to program the FPGA so that it can generate a proper stimulus voltage that being applied thereby increasing the range of the voltage being applied.

7. A method of embedded firmware and system control modules that allow for control, calibration and instantaneous communications between the software modules on the GUI of the host computer and the microprocessor and computer on the a data acquisition board.

8. A system of placing two probes in any culture dish to monitor the signals being produced by the cells, placing a non-invasive charge on the probe that will vary on both the frequency and applied voltage depending on the cell type being interrogated.
